(19) United States
(12) Patent Application Publication
(10) Pub. No.: US 2006/0240014 A1
(43) Pub. Date: Oct. 26, 2006

(54) Methods and compounds for the treatment of vascular stenosis

(75) Inventor: Vikas P Sukhatme, Newton, MA (US)

Correspondence Address: CLARK & ELBING LLP, 101 FEDERAL STREET, BOSTON, MA, 02110, US

(73) Assignee: Beth Israel Deaconess Medical Center, Boston, MA (US)

(21) Appl. No.: 10/559,057

(22) PCT Filed: Jun. 1, 2004
(86) PCT No.: PCT/US04/17273

§ 371 Date: May 30, 2006

Related U.S. Application Data
(60) Provisional application No. 60/475,295, filed on Jun. 3, 2003.

(51) Int. Cl. A61K 39/395; A61K 38/22; A61K 31/505; A61K 31/573; A61K 31/522; A61K 31/4745; A61K 31/401; A61K 31/366; A61K 31/22; A61K 31/355; A61K 31/5377

(52) U.S. Cl. 424/145.1; 514/275; 514/291; 514/171; 514/12; 514/154; 514/406; 514/423; 514/460; 514/548; 514/263.31; 514/458; 514/284; 514/251; 514/232.5; 514/11

(57) ABSTRACT

This invention features a method of treatment for vascular stenosis or restenosis using a combination of N-phenyl-2-pyrimidine derivatives such as imatinib mesylate and PI3K inhibitors, such as rapamycin.

### BACKGROUND OF THE INVENTION

The invention relates to methods and compositions for the treatment of vascular stenosis or restenosis.

Coronary artery disease (CAD) is the most common cause of morbidity and mortality in the United States, affecting some 7 million Americans. CAD is often the result of stenosis, or the narrowing of the arterial lumen, thereby reducing or totally blocking the blood supply to the heart muscles. Stenosis begins in the intima of the artery with the deposition of fatty debris from blood. Smooth muscle cells from the internal elastic membrane and media proliferate into the intima. Collagen and elastin produced from these cells accumulate resulting in a fibrous plaque. As the process continues, cholesterol rich material and necrotic cells accumulating in the plaque cause it to encroach upon the arterial lumen. Eventually, the plaque calcifies and hardens. The narrowed lumen of the artery does not permit adequate blood flow causing that portion of the myocardium to become ischemic. An advanced plaque may rupture or platelets may aggregate at the site to produce an intravascular blood clot or thrombus. A sudden critical reduction in blood supply to the myocardium, usually because of plaque rupture and/or thrombosis, leads to acute myocardial infarction.

Treatment of the stenosed artery usually involves one of two options: by-pass surgery or percutaneous transluminal angioplasty (PTA). Although effective in providing an alternate route for blood flow, by-pass surgery is a high-risk and high-cost procedure. In contrast, PTA is a safer, less intrusive, and less expensive method of treatment.

PTA, commonly known as angioplasty, has proven to be a successful method of treatment for opening the blocked, or stenosed, vessel and restoring blood flow. However, it has been found that restenosis, or re-narrowing of the vessel lumen, frequently occurs. In the case of coronary angioplasty, restenosis occurs in approximately 20-50% of cases within six months of the procedure. An analysis of insurance claims in 1993 estimated the cost of treating restenosis alone to be $1.6 billion annually.

Restenosis is believed to result from the initiation of the wound healing process that occurs after balloon injury to the arterial endothelium during angioplasty. The endothelial injury allows for platelet adhesion and aggregation and the release of growth factors leading to proliferation and extracellular matrix synthesis by smooth muscle cells that have migrated in response to the injury. The end result of this process is neointimal hyperplasia and a re-narrowing of the arterial lumen.

Imatinib mesylate (Gleevec®) is an N-phenyl-2-pyrimidine derivative, which has been shown to inhibit Bcr-Abl tyrosine kinase activity as well as c-kit, c-Abl and PDGFR tyrosine kinase activity. Clinical trials using imatinib mesylate to treat patients with chronic myelogenous leukemia have shown a great deal of success.

PI3K is a family of lipid kinases which can phosphorylate the 3′ hydroxyl group of inositol phospholipids to stimulate a number of cellular signaling pathways involved in cell growth and proliferation. One of the PI3K pathways known to be involved in cell cycle regulation and proliferation is the PI3K-Akt-mTOR pathway. PI3K pathway inhibitory compounds, such as rapamycin, which inhibit the activity of specific proteins along this pathway, would likely block cell proliferation and have shown promise as anti-cancer therapeutics.

### SUMMARY OF THE INVENTION

The present invention provides methods to treat vascular stenosis or restenosis following angioplasty. These methods feature the use of compounds, such as N-phenyl-2-pyrimidine derivatives (e.g., imatinib mesylate), to inhibit the biological activity of the platelet derived growth factor receptor (PDGFR), in combination with a phosphoinositide 3-kinase (PI3K) pathway inhibitor compound (e.g., rapamycin).

Specifically, the invention relates to the use of N-phenyl-2-pyrimidine derivatives of the formula: 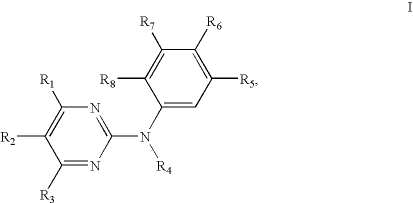
where R₁ is hydrogen or C₁-C₃ alkyl; R₂ is hydrogen or C₁-C₃ alkyl, R₃ is 2-pyridyl, 3-pyridyl, 4-pyridyl, 2-methyl-3-pyridyl, 4-methyl-3-pyridyl, 2-furyl, 5-methyl-2-furyl, 2,5-dimethyl-3-furyl, 2-thienyl, 3-thienyl, 5-methyl-2-thienyl, 2-phenothiazinyl, 4-pyrazinyl, 2-benzofuryl, N-oxido-2-pyridyl, N-oxido-3-pyridyl, N-oxido-4-pyridyl, 1H-indol-2-yl, 1H-indol-3-yl, 1-methyl-1H-pyrrol-2-yl, 4-quinolinyl, 1-methyl-pyridinium-4-yliodide, dimethylaminophenyl, or N-acetyl-N-methyl-aminophenyl; R₄ is hydrogen, C₁-C₃ alkyl, —CO—CO—O—C₂H₅, or N,N-dimethylaminoethyl; at least one of R₅, R₆, R₇, and R₈ is C₁-C₆ alkyl, C₁-C₃ alkoxy, chloro, bromo, iodo, trifluoromethyl, hydroxy, phenyl, amino, mono(C₁-C₃-alkyl)amino, di(C₁-C₃ alkyl)amino, C₂-C₄ alkanoyl, propenyloxy, carboxy, carboxymethoxy, ethoxycarbonylmethoxy, sulfanilamido, N,N-di(C₁-C₃ alkyl)sulfanilamido, N-methylpiperazinyl, piperidinyl, 1H-imidazol-1-yl, 1H-triazol-1-yl, 1H-benzimidazol-2-yl, 1-naphthyl, cyclopentyl, 3,4-dimethylbenzyl, or a radical of one of the formulae:
—CO₂R, —NHC(═O)R, —N(R)CH₂C(═O)R, —O(CH₂)ₙN(R)R, —CH₂(C═O)NH(CH₂)ₙN(R)R, —CH(CH₃)NHCHO, —CH(CH₃)C—N—OH, —CH(CH₃)C═N—O—CH₃, —CH(CH₃)NH₂, —NHCH₂CH₂(C═O)N(R)R, 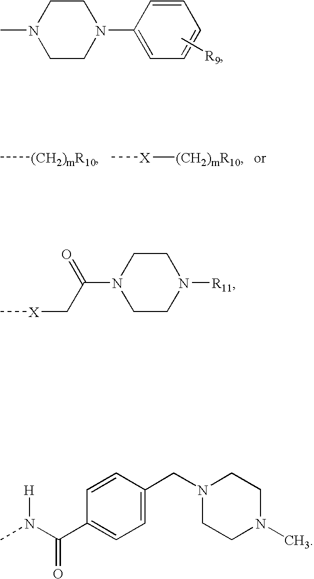
where R is C₁-C₃ alkyl, X is oxygen, or sulfur, m is 1, 2, or 3, n is 2 or 3; R₉ is hydrogen, C₁-C₃ alkyl, C₁-C₃ alkoxy, chloro, bromo, iodo, or trifluoromethyl; R₁₀ is 1H-imidazol-1-yl or morpholinyl; and R₁₁ is C₁-C₃ alkyl or unsubstituted phenyl, or phenyl that is monosubstituted by C₁-C₃ alkyl, halogen or trifluoromethyl; and the remaining of the substituents R₅, R₆, R₇, and R₈ are hydrogen; or a pharmaceutically acceptable salt of the N-phenyl-2-pyrimidine compound containing at least one salt-forming group where the compound inhibits PDGFR biological activity and the administering is in a dose sufficient to prevent or treat vascular stenosis or restenosis following angioplasty.

The compounds of Formula I and their preparation are disclosed in EP-A-0 233 461 and in U.S. Pat. Nos. 4,876,252; 5,516,775; 5,705,502; and 5,521,184, incorporated herein by reference.

In one preferred embodiment, the compound is of the formula

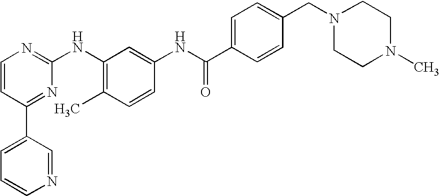

This compound is hereafter referred to as imatinib mesylate (also known as Gleevec™).

In one aspect, the invention features a method for preventing or treating the occurrence of vascular stenosis or restenosis following angioplasty. The method involves administering to a patient (i) a first compound capable of inhibiting platelet derived growth factor receptor (PDGFR) biological activity and (ii) a PI3K pathway inhibitor compound, where the administering is in an amount and for a time sufficient to prevent or reduce the occurrence of stenosis or restenosis following angioplasty. In one embodiment, the first compound is an N-phenyl-2-pyrimidine derivative (e.g., imatinib mesylate). In another embodiment, the first compound inhibits PDGFR β biological activity. In yet another embodiment, the compound inhibits PDGFR biological activity stimulated by a PDGF-BB ligand. In yet another embodiment, the PI3K pathway inhibitor compound inhibits the activity of any protein on the PI3K/Akt/mTOR signaling pathway. In one preferred embodiment, the compound (e.g., rapamycin) inhibits the activity of mTOR. In various embodiments, the restenosis is characterized by the migration of smooth muscle cells into the intima, by the proliferation of vascular smooth muscle cells, or by the deposition of extracellular matrix. In another preferred embodiment, the vascular stenosis is treated with angioplasty and the use of a stent. In one preferred embodiment, the restenosis follows angioplasty and the use of a stent for treatment. In another embodiment, the stent is coated with a compound capable of inhibiting PDGFR biological activity and a PI3K pathway inhibitor compound. In another preferred embodiment, the compound capable of inhibiting PDGFR biological activity is imatinib mesylate and the PI3K pathway inhibitor compound is rapamycin. In yet another embodiment, the stent is coated with a compound capable of inhibiting PDGFR biological activity and a PI3K pathway inhibitor compound. In yet another embodiment, the compound capable of inhibiting PDGFR biological activity and the PI3K pathway inhibitor compound are given in combination with a pharmaceutically acceptable carrier. In another embodiment, the amount is sufficient to prevent or reduce vascular smooth muscle cell hyperplasia. In yet another preferred embodiment, the method further involves administering to a patient at least one compound selected from the group consisting of an angiogenesis inhibitor, an anti-proliferative compound, an immunosuppressive compound, an anti-migratory compound, an anti-platelet agent, and an anti-fibrotic compound.

In a related aspect, the invention features a pharmaceutical composition containing (i) a compound capable of inhibiting PDGFR biological activity and (ii) a PI3K pathway inhibitor compound. In one preferred embodiment, the pharmaceutical composition that further contains at least one additional compound selected from the group consisting of any one or more of the following: an angiogenesis inhibitor, an anti-proliferative compound, an immunosuppressive compound, an anti-migratory compound, an anti-platelet agent, and an anti-fibrotic compound. In preferred embodiments, the first compound is an N-phenyl-2-pyrimidine derivative (e.g., imatinib mesylate).

In another aspect, the invention features a kit containing (i) a compound capable of inhibiting PDGFR biological activity, (ii) a PI3K pathway inhibitor compound, and (iii) an additional compound that is selected from the group consisting of: an angiogenesis inhibitor, an anti-proliferative compound, an immunosuppressive compound, an anti-migratory compound, an anti-platelet agent, and an anti-fibrotic compound; and (iii) instructions for administering the compound capable of inhibiting PDGFR biological activity and the second compound to a patient diagnosed with or at risk of developing stenosis or restenosis following angioplasty. In one embodiment, the PDGFR inhibitor is N-phenyl-2-pyrimidine derivative (e.g., imatinib mesylate). In preferred embodiments, the stenosis or restenosis is characterized by the migration of smooth muscle cells into the intima, by the proliferation of vascular smooth muscle cells, or by the deposition of extracellular matrix. In other preferred embodiments, the additional compound is selected from the group consisting of: an angiogenesis inhibitor, an anti-proliferative compound, an immunosuppressive compound, an anti-migratory compound, an anti-platelet agent, and an anti-fibrotic compound.

Non-limiting examples of angiogenesis inhibitors include any one or more of the following: an antibody (e.g., an antibody that binds VEGF-A, an antibody that binds a vascular endothelial growth factor VEGF receptor and blocks VEGF binding), avastin, endostatin, angiostatin, restin, tumstatin, TNP-470, 2-methoxyestradiol, thalidomide, a peptide fragment of an anti-angiogenic protein, canstatin, arrestin, a VEGF kinase inhibitor, CPTK787, SFH-1, an anti-angiogenic protein, thrombospondin-1, platelet factor-4, interferon-α, an agent that blocks TIE-1 or TIE-2 signalling, or PIH12 signalling, an agent that blocks an extracellular vascular endothelial (VE) cadherin domain, an antibody that binds to an extracellular VE-cadherin domain, tetracycline, penicillamine, vinblastine, cytoxan, edelfosine, tegafur or uracil, curcumin, green tea, genistein, resveratrol, N-acetyl cysteine, captopril, a cox-2 inhibitor, celecoxib (e.g., CELEBREX), and rofecoxib (e.g., VIOXX).

In other preferred embodiments of any of the previous aspects, an anti-proliferative compound include any one or more of the following: rapamycin, taxol, troglitazone, an antibody that binds bFGF, an antibody that binds bFGF-saporin, a statin, an ACE inhibitor, suramin, 17 beta-estradiol, atorvastatin, fluvastatin, lovastatin, pravastatin, simvastatin, cerivastatin, perindopril, quinapril, captopril, captopril, lisinopril, enalapril, fosinopril, cilazapril, ramipril, and a kinase inhibitor.

Non-limiting examples of an immunosuppressive compound include any one or more of the following: prednisone, FTY720, methylprednisolone, α-tocopherol, azathioprine, chlorambucil, cyclophosphamide, an antibody that binds to an IL-2 receptor or to CTLA4, methotrexate, mycophenolate mofetil, cyclosporine, an agent that interferes with macrophage function, an agent that inhibits P-selectin PSGL-1, VLA-4, VCAM-1 or that blocks that Mac-1 biological function, and FTY720.

Non-limiting examples of an anti-migratory compound include any one or more of the following cyproheptadine, methysergide, bosentan, YM087, cyproheptadine, ketanserin, and anplag.

Non-limiting examples of an anti-platelet agent include any one or more of the following ticlopidine, cilostazol, dipyridamole, abciximab, clopidogrel, dipyridimole, a glycoprotein iib/iiia inhibitor, eptifibatide, tirofiban, and a phosphodiesterase III inhibitor.

Non-limiting examples of an anti-fibrotic compound include any one or more of the following: an agent that blocks transforming growth factor beta (TGF-β) signaling or inhibits activation of plasminogen activator inhibitor-1 promoter activity, an antibody that binds to TGF-β or to a TGF-β receptor, an antibody that binds to TGF-β receptor I, II, or III, a kinase inhibitor, an agent that blocks connective tissue growth factor (CTGF) signaling, an agent that inhibits prolyl hydroxylase, an agent that inhibits procollagen C-proteinase, pirfenidone, silymarin, pentoxifylline, colchicines, Embrel (etanercept), Remicade (infliximab), an agent that antagonizes TGF-β, an agent that antagonizes or inhibits CTGF, and an agent that inhibits VEGF.

It should be noted that many compounds possess more than one activity. For example, a serotonin receptor antagonist can have both anti-migratory and anti-proliferative activity. These categories are not meant to limit the compounds, but to provide broad descriptions of the potential activities of each compound.

By “aryl” is meant a carbocyclic aromatic ring or ring system. Unless otherwise specified, aryl groups are from 6 to 18 carbons. Examples of aryl groups include phenyl, naphthyl, biphenyl, fluorenyl, and indenyl groups.

By “heteroaryl” is meant an aromatic ring or ring system that contains at least one ring hetero-atom (e.g., O, S, N). Unless otherwise specified, heteroaryl groups are from 1 to 9 carbons. Heteroaryl groups include furanyl, thienyl, pyrrolyl, imidazolyl, pyrazolyl, oxazolyl, isoxazolyl, thiazolyl, isothiazolyl, triazolyl, oxadiazolyl, oxatriazolyl, pyridyl, pyridazyl, pyrimidyl, pyrazyl, triazyl, benzofuranyl, isobenzofuranyl, benzothienyl, indole, indazolyl, indolizinyl, benzisoxazolyl, quinolinyl, isoquinolinyl, cinnolinyl, quinazolinyl, naphtyridinyl, phthalazinyl, phenanthrolinyl, purinyl, and carbazolyl groups.

By “heterocycle” is meant a non-aromatic ring or ring system that contains at least one ring heteroatom (e.g., O, S, N). Unless otherwise specified, heterocyclic groups are from 1 to 9 carbons. Heterocyclic groups include, for example, dihydropyrrolyl, tetrahydropyrrolyl, piperazinyl, pyranyl, dihydropyranyl, tetrahydropyranyl, tetrahydrofuranyl, dihydrothiophene, tetrahydrothiophene, and morpholinyl groups.

By “halide” or “halogen” or “halo” is meant bromine, chlorine, iodine, or fluorine.

Aryl, heteroaryl, or heterocyclic groups may be unsubstituted or substituted by one or more substituents selected from the group consisting of C₁₋₆ alkyl, hydroxy, halo, nitro, C₁₋₆ alkoxy, C₁₋₆ alkylthio, trifluoromethyl, C₁₋₆ acyl, arylcarbonyl, heteroarylcarbonyl, nitrile, C₁₋₆ alkoxycarbonyl, arylalkyl (wherein the alkyl group has from 1 to 6 carbon atoms), and heteroarylalkyl (wherein the alkyl group has from 1 to 6 carbon atoms).

By “angioplasty” or “percutaneous transluminal angioplasty (PTA)” is meant any percutaneous transluminal method of decreasing stenosis within a blood vessel, whether caused by the existence of an atheromatous plaque, thrombosis, embolus, and/or mineral deposit, by any of a number of means such as balloon dilation, thermal ablation, laser atherectomy, mechanical shaving, extraction, or ultrasonic pulverization. Examples include coronary angioplasty, also known as PTCA, and angioplasty used to treat peripheral vascular disease such as femoropopliteal angioplasty.

By “anti-fibrotic agent” is meant any agent, which can reduce or inhibit the production of extracellular matrix components including but not limited to fibronectin, proteoglycan, collagen, and elastin. Examples of anti-fibrotic agents include, but are not limited to, antagonists of TGFβ and CTGF.

By “anti-migratory compound” is meant any compound that blocks the movement or migration of smooth muscle cells. Anti-migratory compounds also include any compound that can inhibit any of the cellular signaling proteins known to induce migration of smooth muscle cells. Examples of compounds with anti-migratory activity include, but are not limited to, serotonin (e.g., 5-HT₂) receptor antagonists (e.g., cyproheptadine or methysergide), compounds that antagonize either of the endothelin-1 receptors, ET_{A} and ET_{B}, (e.g., bosentan), and vasopressin receptor antagonists (e.g., YM087).

By “anti-platelet agent” is meant any compound that can inhibit one or more of the steps leading to platelet activation (i.e., platelet shape change, secretion of platelet granule contents, and aggregation of platelets). Preferably these compounds will inhibit the production of PDGF. Examples of anti-platelet agents include, but are not limited to, cyclooxygenase inhibitors (e.g., aspirin), ADP inhibitors (e.g., ticlopidine and clopidogrel), phophodiesterase III inhibitors (e.g., cilostazol and dipyridamole), and glycoprotein IIB/IIIA inhibitors (e.g., abciximab).

By “anti-proliferative compound” is meant any compound, which can reduce or inhibit the proliferation of vascular smooth muscle cells or endothelial cells. Anti-proliferative compounds include any compound that can inhibit any of the cellular signaling proteins known to induce proliferation of smooth muscle cells. Examples of anti-proliferative compounds include, but are not limited to, bFGF inhibitors, statins, ACE inhibitors, suramin, paclitaxel (taxol), 17 beta-estradiol, and troglitazone.

By “effective amount” is meant an amount sufficient to prevent or ameliorate vascular stenosis or restenosis following angioplasty. It will be appreciated that there will be many ways known in the art to determine the effective amount for a given application. For example, the pharmacological methods for dosage determination may be used in the therapeutic context.

By “immunosuppressive compound” is meant any agent that can reduce or inhibit the natural immune response induced by chemical, biological, or physical agents. Preferably, an immunosuppressive compound can inhibit monocyte or macrophage activity in any one of four ways: 1) by decreasing the production or increasing the removal of monocytes, 2) by preventing differentiation of monocytes into macrophages, 3) by preventing monocytes from migrating into the region of hyperplasia, and 4) by blocking the activation of macrophages. By macrophage activity is meant the ability to present foreign antigen to antigen-reactive lymphocytes and the ability to induce both the humoral and cell-mediated immune responses.

Non-limiting examples of preferred immunosuppressive compounds include steroids (e.g., prednisone, methylprednisolone) and FTY720 (e.g., Shuurman et al., Transplantation 74:951-960, 2002; Droogan et al., Neurology 50:224-229, 1998). Additional exemplary immunosuppressive compounds include antibodies or compounds that block the production or functioning of mac-1, an antigen on macrophages known to be important for transmigration (e.g., Eslami et al., J. Vasc. Surg. 34:923-929, 2001, Shang et al., Eur. J. Immunol. 28:1970-1979, 1998), any compound that blocks monocyte chemoattractant protein (MCP-1) production or function or both (Ikeda et al., Clin. Cardiol. 25:143-147, 2002), antioxidants such as alpha-tocopherol (Devaraj et al., Nutr. Rev. 60:8-14, 2002; Terasawa et al., Biofactors 11:221-233, 2000), as well as compounds that block P-selectin, PSGL-1, VLA-4 or VCAM-1 activity (Huo et al., Acta Physiol. Scand. 173:35-43, 2001).

By “migration” is meant the movement of cells from one area of a vessel to another. In particular, migration refers to the movement of smooth muscle cells in vivo from the medial layers of a vessel into the intima.

By “neointimal hyperplasia” is meant an abnormal proliferation of smooth muscle cells after migration into the intima. Abnormal as used herein means division or growth of cells, but not cancer cells, that occurs more rapidly or to a significantly greater extent than typically occurs in a normally functioning cell of the same type.

By “pharmaceutically acceptable carrier” is meant a carrier that is physiologically acceptable to the treated mammal while retaining the therapeutic properties of the compound with which it is administered. One exemplary pharmaceutically acceptable carrier substance is physiological saline. Other physiologically acceptable carriers and their formulations are known to one skilled in the art and described, for example, in Remington's Pharmaceutical Sciences, (20^{th} edition), ed. A. Gennaro, 2000, Lippincott, Williams & Wilkins, Philadelphia, Pa.

By “PI3K pathway” is meant any cell-signaling pathway that is initiated by a signaling event from a phosphoinositide 3-kinase (PI3K) family member. PI3K family members phosphorylate phosphoinositides at the 3-hydroxyl position. The PI3Ks are involved in a large number of cellular processes, including apoptosis, proliferation, cell motility, and adhesion. Downstream targets of PI3K signaling pathways are numerous and diverse and many are still as yet unidentified. Although any and all of these various signaling pathways and substrate proteins are included, the preferred pathway is the PI3K-Akt-mTOR signaling pathway which is responsible for activating gene transcription, cell cycle entry, and cell proliferation. See Blume-Jensen and Hunter (Nature, 411:355-365, 2001) for a review of the various PI3K signaling pathways. By “PI3K pathway inhibitor compound” is meant any compound which can reduce or attenuate any activity of PI3K or the downstream signaling proteins that are activated by PI3K activity. An inhibitor of a PI3K pathway will preferably target the kinase activity of PI3K and can also inhibit PI3K interactions with other proteins. Examples of PI3K inhibitors include wortmannin and LY294002. For a review of PI3K inhibitors see Walker et al. (Molecular Cell, 6:909-919, 2000). Akt, a downstream signaling protein, is also a kinase and inhibitors of this molecule will preferably reduce or eliminate its kinase activity or block protein-protein interactions. mTOR, another downstream signaling protein also known as FK506 binding protein, is a kinase which phosphorylates proteins including p70^{s6k}. Preferred inhibitors of mTOR will inhibit kinase activity or block protein-protein interactions. One example of a novel mTOR inhibitor is AP23573 which is currently under development by Ariad Pharmaceuticals. A preferred example of an mTOR inhibitor is rapamycin. There are numerous references in the literature describing the use of rapamycin including Grunwald et al., (Cancer Res., 62:6141-6145, 2002), Morales J M (Kidney Int. Suppl., 82:81-87, 2002), Kenerson et al. (Cancer Res., 62:5645-5650, 2002), Cotterell et al. (Clin. Trasplant., 16 suppl. 7:49-51, 2002), and Pene et al., (Oncogene, 21:6587-6597, 2002).

By “platelet derived growth factor receptor (PDGFR) biological activity” is meant any and all of the functions of the PDGFR. The PDGFR functions to elicit a mitogenic response as well as a chemotactic response in cells. Its functions can include: ligand binding which can include any of three dimeric forms of the PDGF ligand (AA, AB, or BB), receptor dimerization, autophosphorylation on a tyrosine residue, transphosphorylation of a substrate polypeptide or protein on a tyrosine residue, and recruitment of SH2 domain containing proteins. There are many standard assays for PDGFR biological activity known in the art and any of these can be used to assay a potential compound for its ability to inhibit PDGFR biological activity. Examples include cell proliferation assays such as BrdU labeling and cell counting experiments; quantitative assays for DNA synthesis such as ³H-thymidine incorporation; ligand binding assays and Scatchard plot analysis; receptor dimerization assays; and cellular phosphorylation assays (see, for example, Bioukar et al., J. Biol. Chem. 274:21457-63, 1999; Conway et al., Biochem. J. 337:171-7, 1999; Vignais et al., Mol. Cell Biol. 19:3727-35, 1999; Baxter et al., J. Biol. Chem. 273:17050-5, 1998; DeMali et al., Mol. Cell Biol. 18:2014-22; and Davies et al., Circ. Res. 86:779-786, 2000). One preferred assay for PDGFR biological activity is a phosphorylation assay using anti-phosphotyrosine antibodies (e.g., 4G10, Upstate Biotechnology Inc.). Immunoblots on whole cell lysates can be performed using this antibody to assay for an overall increase in cellular tyrosine phosphorylation. Tyrosine phosphorylation of specific substrates of PDGFR, such as Src or p42/p44 MAP kinase, can also be analyzed by immunoprecipitating the substrate protein and immunoblotting using an anti-phosphotyrosine antibody. Autophosphorylation of PDGFR itself can also be measured by immunoprecipitating PDGFR and immunoblotting with an anti-phosphotyrosine antibody. Each of the above-mentioned assays can be quantitated and used to determine the effects of a potential inhibitor of PDGFR biological activity as compared to a control compound that does not affect PDGFR biological activity. Inhibition of biological activity depends on the assay being used but generally connotes a reduction of at least 10% of the assayed activity, preferably at least 25%, more preferably at least 50%, and most preferably at least 75% of the assayed activity as compared to a control.

By “prevent or reduce” is meant a reduction in the narrowing of the vessel lumen diameter such that the blood flow does not fall below values considered to be normal for the specific vessel. Clinicians or practitioners skilled in the art will be familiar with the normal values for blood flow for a specific vessel. As used herein “prevent or reduce” can also be used in reference to neointimal hyperplasia and includes any decrease of 20% or greater (more preferably 50% or greater, most preferably 75% or greater) in the proliferation rate or overall number of vascular smooth muscle cells. As used herein “prevent or reduce” can also mean a reduction in the narrowing of the vessel lumen diameter such that the diameter of the lumen after treatment is 0 to 25%, preferably 25 to 50%, and most preferably 50% or more than the diameter of the lumen before treatment.

By “proliferation” is meant an increase in cell number, i.e., by mitosis of the cells. As used herein proliferation does not refer to neoplastic cell growth.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

By “restenosis” is meant a re-narrowing or blockage of an artery at the same site where treatment, such as an angioplasty or stent procedure, has already taken place. If restenosis occurs within a stent that has been placed in an artery, it is technically called “in-stent restenosis,” the end result being a narrowing in the artery caused by a build-up of substances that may eventually block the flow of blood. Restenosis is histologically similar to vascular stenosis and is characterized by the appearance of cells and matrix in the intimal layer of the artery and concentric depression of the outer layer of the blood vessel (Garas et al., Pharma. & Ther., 92:165-178, 2001).

By “smooth muscle cells” is meant those cells derived from the medial layers of vessels and adventitial vessels. Characteristics of smooth muscle cells include a histological morphology (under light microscopic examination) of a spindle shape with an oblong nucleus located centrally in the cell with nucleoli present and myofibrils in the sarcoplasm. Under electron microscopic examination, smooth muscle cells have long slender mitochondria in the juxta-nuclear sarcoplasm, a few tubular elements of granular endoplasmic reticulum, and numerous clusters of free ribosomes. A small Golgi complex may also be located near one pole of the nucleus. The majority of the sarcoplasm is occupied by thin, parallel myofilaments that may be, for the most part, oriented to the long axis of the muscle cell. These actin-containing myofibrils may be arranged in bundles with mitochondria interspersed among them. Scattered through the contractile substance of the cell may also be oval dense areas, with similar dense areas distributed at intervals along the inner aspects of the plasmalemma.

By “stenosis” is meant a pathologic narrowing of a blood vessel.

By “stent” is meant a slender thread, rod, or catheter lying within the lumen of a vessel used to provide support and to assure patency of an intact but contracted lumen.

By “thrombosis” is meant the formation or presence of a clot in the cardiovascular system that may be occlusive or attached to the vessel without obstructing the lumen.

By “tyrosine kinase activity” is meant the ability to catalyze the transfer a phosphate group from adenosine triphosphate (ATP) to a tyrosine residue on a substrate polypeptide or protein.

Other features and advantages of the invention will be apparent from the following description of the preferred embodiments thereof, and from the claims.

### BRIEF DESCRIPTION OF THE DRAWINGS

FIG. 1 shows the dose response inhibition of hPDGF-BB (20 ng/mL) induced migration of human aortic vascular smooth muscle cells by Gleevec alone (G), rapamycin alone (R), and combinations of Gleevec and rapamycin. Drug concentrations for G are in ng/mL and for R are in pg/mL. The first lane has no PDGF added and the first two lanes have no drug compounds added. The results are expressed as means±S.E.M. of triplicate readings.

### DETAILED DESCRIPTION

Coronary artery disease is a major worldwide health problem and is usually caused by vascular stenosis. PTA, also known as angioplasty, is widely used to treat patients with symptomatic CAD or vascular stenosis. Although often initially successful, an angioplasty procedure is frequently complicated by restenosis within six months in 20-50% of the procedures. There is no known cure available for the treatment of this costly limitation of angioplasty therapy. N-phenyl-2-pyrimidine derivatives such as imatinib mesylate function as tyrosine kinase inhibitors, which have proven very effective in the treatment of diseases such as chronic myelogenous leukemia. Rapamycin is an inhibitor of the PI3K/Akt/mTOR pathway which is known to have anti-proliferative effects. This invention features a method of treatment for vascular stenosis or restenosis using a combination of N-phenyl-2-pyrimidine derivatives such as imatinib mesylate and PI3K inhibitors such as rapamycin. By preventing mitogenic signaling from the PDGFR and blocking cellular proliferation, the hyperproliferation of vascular smooth muscle cells is inhibited. This inhibition results in a decrease in neointimal hyperplasia thereby preventing or reducing the occurrence of vascular stenosis or restenosis.

While the detailed description presented herein refers specifically to the N-phenyl-2-pyrimidine derivative imatinib mesylate, it will be clear to one skilled in the art that the detailed description can also apply to any N-phenyl-2-pyrimidine derivatives such as those described in U.S. Pat. Nos. 4,876,252; 5,516,775; 5,705,502; and 5,521,184, incorporated herein by reference, or any other PDGFR inhibitors. While the detailed description presented herein refers specifically to rapamycin as the PI3K pathway inhibitor, it will be clear to one skilled in the art that the detailed description can also apply to any inhibitor of PI3K or downstream effector molecules such as Akt or mTOR.

Therapeutic Uses

The invention features methods for treating vascular stenosis or restenosis associated with angioplasty by administering a combination of N-phenyl-2-pyrimidine derivatives, such as imatinib mesylate, and PI3K/Akt/mTOR pathway inhibitors such as rapamycin. Imatinib mesylate and rapamycin may be administered within six months, two months, one month, fourteen days, ten days, five days, twenty-four hours, or one hour of each other. Preferably, imatinib mesylate and rapamycin are administered simultaneously. Imatinib mesylate and rapamycin are administered with a pharmaceutically acceptable diluent, carrier, or excipient, in unit dosage form. Administration may be parenteral, intravenous, subcutaneous, oral, or local at the site of the arterial injury.

The composition can be in the form of a pill, tablet, capsule, liquid, or sustained release tablet for oral administration; or a liquid for intravenous, subcutaneous or parenteral administration; or a polymer or other sustained release vehicle for local administration.

Methods well known in the art for making formulations are found, for example, in “Remington: The Science and Practice of Pharmacy” (20th ed., ed. A. R. Gennaro AR., 2000, Lippincott Williams & Wilkins, Philadelphia, Pa.). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. The concentration of the compound in the formulation will vary depending upon a number of factors, including the dosage of the drug to be administered, and the route of administration.

The compound may be optionally administered as a pharmaceutically acceptable salt, such as a non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

For oral use of imatinib mesylate dosages range from 50 mg to 5000 mg per day, more preferably 100 mg to 1000 mg per day, and most preferably 100 mg to 800 mg per day given in one daily dose, two daily doses, or up to four daily doses. For rapamycin, dosages are provided to produce a blood level of rapamycin that ranges from 0.001 μg/ml to 10 μg/ml with a preferred range of 0.005 μg/ml to 0.1 μg/ml.

For local administering, the compound can be directly injected at the site of angioplasty. In a preferred embodiment of the invention a stent coated with imatinib mesylate or rapamycin or both will be inserted in order to mechanically provide support to the vessel and to deliver the anti-proliferative compounds to the site of injury.

Rapamycin and imatinib mesylate can be given in any combination of oral, systemic, and local administering that proves to be most effective. For example, both compounds can be given orally, both can be given locally, both can be given systemically, or one can be given orally while one is given locally or systemically.

The timing of administering any of the compounds of the present invention will depend on various clinical factors including the overall health of the patient, the diagnosis of CAD, and the relative risk of myocardial infarction. In general, the drug compounds can be administered prior to, during, after, or any combination thereof, the angioplasty procedure. The drug compounds can also be administered prior to, during, after, or any combination thereof, an angioplasty procedure in which a stent is also inserted. The drug compounds can be administered at the time the stent is inserted, at a later time when “in-stent restenosis” is detected, or both. Administration of the drug compounds prior to, during, and/or after angioplasty and stent insertion can be systemic or local.

In general, one dosage of each of the compounds is given one week prior to the angioplasty procedure, and can be continued for a period of time ranging from −7 to 365 days, preferably −7 to 180 days, more preferably −7 to 120 days, and most preferably −7 to 90 days where day 0 is the day of the angioplasty procedure. Accordingly, −7 days would be 7 days prior to the day of angioplasty. Repeated applications of the local administration can be required and will include repeated injections, applications of the compounds or replacement of the drug-coated stent.

Stents

A stent acts as a scaffold to provide structural support for a vessel. A stent, or a small, expandable wire tube, is often used in the treatment of coronary artery disease. During angioplasty, the balloon is placed inside the stent and inflated, and this opens the stent and pushes it into place against the artery wall. The stent is left permanently and often, because the stent is meshlike, the cells lining the blood vessel grow through and around the stent to help secure it. Stents are commonly used in angioplasty to restore and maintain adequate blood flow to the heart and to prevent the artery wall from collapsing or closing again.

In the present invention, a method is provided for preventing onset of or reducing risk of restenosis following angioplasty, using a stent coated with a therapeutically effective amount of imatinib mesylate or rapamycin or both and any other compound where desired.

Stents are coated using standard methods known in the art. Methods for coating stents are generally known and examples can be found in U.S. Pat. Nos. 6,153,252; 6,258,121; and 5,824,048, herein incorporated by reference. For each of the therapeutic compounds listed in the present application, the amount of therapeutic agent used will be dependent upon the particular drugs employed. Typically, the amount of drug represents about 0.001% to about 70%, more typically about 0.001% to about 60%, most typically about 0.001% to about 45% by weight of the coating.

Use of Additional Compounds

As described above, the cellular signaling pathways that initiate and sustain neointimal hyperplasia resulting in vascular stenosis and occlusion are numerous and varied. The upstream stimulants can include mitogens and cytokines including platelets, neutrophils or macrophages. Classic proliferation signaling pathways and cellular migration signaling pathways, as well as angiogenic signaling pathways, immunologic response pathways, extracellular matrix deposition, and cell adhesion pathways are also up regulated. The cellular proteins that regulate each of these aspects of neointimal hyperplasia are diverse. Therefore, it is advantageous to target one or more of these pathways when designing therapeutic approaches to combat vascular stenosis and restenosis. The use of additional compounds directed to the diverse pathways involved can potentiate greater therapeutic success while reducing the toxicity of the compounds and the dosages required. As these stenotic lesions are chronic in nature, the ability to target multiple pathways and to reduce the toxicity of these compounds can extend the long-term efficacy in reducing vascular stenosis and restenosis over that seen in current therapeutic approaches such as balloon angioplasty alone. Furthermore, as each cell type may respond differently to each compound, the use of additional compounds will also help to target one or more of the multiple cell types involved in the stenotic lesion.

The present invention also provides for a pharmaceutical composition comprising an N-phenyl-2-pyrimidine derivative capable of inhibiting PDGFR biological activity, a PI3K pathway inhibitor compound, and, where desired, at least one additional compound selected from the following: (i) an angiogenesis inhibitor, (ii) an anti-proliferative compound, (iii) an immunosuppressive compound, (iv) an anti-migratory compound, (v) an anti-platelet agent, and (vi) an anti-fibrotic compound.

Angiogenesis inhibitors include but are not limited to vascular endothelial growth factor (VEGF) inhibitors such as antibodies against VEGF-A, antibodies against one of the VEGF receptors, and small molecule compounds that inhibit the tyrosine kinase activity of one of the VEGF receptors. Additional examples of angiogenesis inhibitors include endostatin, angiostatin, restin, tumstatin as well as other small molecule inhibitors such as TNP-470, two methoxyestradiol, and thalidomide.

The dosage of the angiogenesis inhibitor will depend on other clinical factors such as weight and condition of the human or animal and the route of administration of the compound. For treating humans or animals, between approximately 0.1 mg/kg to 500 mg/kg body weight of the angiogenesis inhibitor can be administered. A more preferable range is 0.5 mg/kg to 100 mg/kg body weight with the most preferable range being from 1 mg/kg to 50 mg/kg body weight. Depending upon the half-life of the angiogenesis inhibitor in the particular animal or human, the angiogenesis inhibitor can be administered between several times per day to once a week. The methods of the present invention provide for single as well as multiple administrations, given either simultaneously or over an extended period of time.

Anti-proliferative compounds include any compound, which can reduce or inhibit the proliferation of vascular smooth muscle cells or endothelial cells. Examples of anti-proliferative compounds include, but are not limited to, bFGF inhibitors, paclitaxel (taxol), troglitazone, 17 beta-estradiol, ACE inhibitors, statins, and suramins.

The dosage of the anti-proliferative compound depends on clinical factors such as weight and condition of the human or animal and the route of delivery of the compound. In general, for treating humans or animals, between approximately 0.1 mg/kg to 500 mg/kg body weight of the anti-proliferative compound can be administered. A more preferable range is 1 mg/kg to 50 mg/kg body weight with the most preferable range being from 1 mg/kg to 25 mg/kg body weight. Depending upon the half-life of the anti-proliferative compound in the particular animal or human, the compound can be administered between several times per day to once a week. The methods of the present invention provide for single as well as multiple administrations, given either simultaneously or over an extended period of time.

Taxol is administered intravenously at a weekly dosages ranging from approximately 0.5 mg/kg to 5 mg/kg body weight. A more preferable range is 1 mg/kg body weight to 5 mg/kg body weight with the most preferable range being from 1 mg/kg to 2.5 mg/kg body weight. Troglitazone is given orally or intravenously at daily dosages ranging from approximately 0.5 mg/kg to 25 mg/kg body weight. A more preferable range is 1 mg/kg body weight to 20 mg/kg body weight with the most preferable range being from 1 mg/kg to 10 mg/kg body weight.

Statins is the common name for a class of drugs formally known as 3-hydroxy-3-methylglutaryl coenzyme A (HMG CoA) reductase inhibitors. These drugs lower levels of low-density lipoprotein cholesterol. Smooth muscle cell proliferation is a feature of atherogenesis and therefore, drugs affecting this metabolic pathway, such as statins, may reduce smooth muscle cell proliferation. Statins now marketed in the United States include atorvastatin (Lipitor), fluvastatin (Lescol), lovastatin (Mevacor), pravastatin (Pravachol), simvastatin (Zocor), cerivastatin (Baycol, removed from market in August 2001), and a number of other formulations. Dosages for statins may be obtained for each formulation from the pharmaceutical manufacturer. Recommended dosages range from 0.20 to 100 mg daily depending on the particular formulation being used. For example, the recommended daily dosage for fluvastatin is 20 to 40 mg.

Angiotensin-converting enzyme (ACE) inhibitors block the effects of angiotensin II, which is a potent vasoconstrictor. In addition to its vasoconstricting properties, angiotensin also stimulates vascular smooth muscle cell proliferation. ACE inhibitors can be effective at preventing restenosis both through the prevention of vascular recoil and remodeling and the prevention of inflammation and cell proliferation. There are several ACE inhibitors currently marketed in the United States. Examples include perindopril (Aceon), quinapril, captopril (Capoten), Lisinopril (Prinivil, Zestril), enalapril (Vasotec), fosinopril (Monopril), benazepril (Lotensin), cilazapril, and ramipril (Altace). Dosages for ACE inhibitors may be obtained for each formulation from the pharmaceutical manufacturer. Recommended daily dosages range from 1 to 500 mg depending on the particular formulation being used. For example, the preferred dosage for perindopril is 2 mg to 20 mg per day, while the preferred dosage for Capoten is 25 to 150 mg/b.i.d. or t.i.d. with a maximum dosage of 450 mg per day.

Suramin is a polyanionic compound that, after decades of use as an anti-parasitic drug, was recognized for its ability to block autocrine and paracrine growth factors required for the proliferation of smooth muscle cells. Suramin has recently been used in animal studies for the treatment of neoplasms. Suramin is marketed under several brand names including Antrypol and Surmontil. Dosages for suramin may be obtained for each formulation from the pharmaceutical manufacturer. Recommended daily dosages range from 50 to 200 mg depending on the particular formulation being used.

Immunosuppressive compounds include any compounds that can suppress the natural immune response of an animal. Preferred compounds will suppress the activity of monocytes or macrophages or both. Preferably, an immunosuppressive compound can inhibit monocyte and macrophage activity in any one of four ways: 1) by decreasing the production or increasing the removal of monocytes, 2) by preventing differentiation of monocytes into macrophages, 3) by preventing monocytes from migrating into the region of hyperplasia, and 4) by blocking the activation of macrophages. Non-limiting examples of preferred immunosuppressive compounds include antibodies or compounds that block the production or functioning of mac-1, any compound that blocks MCP-1 production or function or both, antioxidants such as alpha-tocopherol, and compounds that block P-selectin, PSGL-1, VLA-4 or VCAM-1. Steroids are another example of a class of immunosuppressive compounds. Non-limiting examples of steroids include prednisone and methylprednisolone. Another example of an immunosuppressive compound is FTY720, which is currently under development by Novartis. Dosages for FTY720 will be determined based on the information gained from Phase II and Phase III clinical trials. Current dosages based on preliminary studies range from 2 mg/day to 5 mg/kg per day.

Anti-migratory compounds include any compound which can reduce or prevent the movement or migration of smooth muscle cells. Specific, non-limiting examples include serotonin receptor (5-HT₂) antagonists, endothelin-1 receptor antagonists, and vasopressin receptor antagonists. Endothelin-1 (ET-1) is a potent vasoconstrictor secreted by endothelial and smooth muscle cells. It has two receptors, ET_{A} located on vascular smooth muscle cells and ET_{B} located on endothelial and vascular smooth muscle cells. Endothelin-1 stimulates vascular smooth muscle cell proliferation and migration alone and in combination with other cytokines. It also stimulates extracellular matrix synthesis. There is some evidence to suggest that both ET-1 and angiotensin II can act synergistically on vascular smooth muscle cells. There is also evidence based on animal studies that ET-1 receptor antagonists and ET-converting enzyme inhibitors are useful in the prevention of neointimal hyperplasia. These data suggest that endothelin-1 receptor antagonists might be useful in the prevention of migration of smooth muscle cells.

Serotonin is secreted mainly by platelets and acts on blood vessels via the 5-HT₁ and 5-HT₂ receptors. Over the past decade, some researchers have suggested that serotonin may be involved in vascular smooth muscle cell proliferation and migration. 5-HT₂ receptor antagonists, Ketanserin and Anplag, were shown to be effective in the prevention of neointimal hyperplasia in rabbit carotid artery balloon angioplasty and vein graft intimal hyperplasia models suggesting that they may be useful in the prevention of proliferation and migration of smooth muscle cells.

Specific examples of anti-migratory compounds include cyproheptadine, bosentan, Ketanserin, Anplag and YM087. Dosages for each compound can be obtained from the manufacturer and will vary depending on the weight and condition of the patient and the route of administration of the compound.

Anti-platelet agents can include any cyclooxygenase inhibitor (e.g., aspirin), ADP inhibitor (e.g., ticlopidine), phophodiesterase III inhibitor (e.g., cilostazol and dipyridamole), or glycoprotein IIB/IIIA inhibitor (e.g., abciximab). Dosages for each compound can be obtained from the manufacturer and will vary depending on the weight and condition of the patient and the route of administration of the compound.

Anti-fibrotic compounds include antagonists of transforming growth factor beta (TGFβ) or connective tissue growth factor (CTGF). The dosage of the anti-fibrotic agent will depend on other clinical factors such as weight and condition of the human or animal and the route of administration of the compound. For treating humans or animals, between approximately 0.1 mg/kg to 500 mg/kg body weight of the anti-fibrotic agent can be administered. A more preferable range is 0.5 mg/kg to 50 mg/kg body weight with the most preferable range being from 1 mg/kg to 25 mg/kg body weight. Depending upon the half-life of the anti-fibrotic agent in the particular animal or human, the anti-fibrotic agent can be administered between several times per day to once a week. The methods of the present invention provide for single as well as multiple administrations, given either simultaneously or over an extended period of time.

It should be noted that although each of the compounds is listed under a specific category of compounds, these categories are not meant to be limiting in scope. Many of the compounds-possess more than one activity and can therefore be included under more than one category. For example, serotonin receptor antagonists can have both anti-proliferative and anti-migratory activity.

For each of the compounds listed, all of the modes of administration described above can be used. As some of the compounds described have shown toxicity when administered orally or systemically, local administration (for example by the coating of a stent inserted into the artery at the time of angioplasty) can also be used. In general, percent composition of the compound will range from 0.05% to 50% weight for weight of compound to coating material used.

The invention also provides for the use of a therapeutically effective amount of a compound capable of inhibiting PDGFR biological activity and rapamycin in combination with radiation therapy. It will be appreciated that there will be many ways known in the art to determine the dosage and duration of treatment. For radiation therapy, gamma rays or beta rays are used in an amount sufficient to induce enough damage to a cell so as to limit its ability to function normally or to destroy the cell altogether. Typical dosages range from 10 to 200 units (Grays). The most common systems used for radiation therapy are either catheter based or radioactive stents. In catheter-based systems, high intensity radioactive sources, in the form of a thin wire or tiny pellets attached to the end of a specially designed catheter are introduced to the site of angioplasty. Irradiation may last for three to five minutes after which the source is retracted. Alternatively, a stent implanted with a small amount of radioactive material is placed permanently at the site of angioplasty. In addition to providing mechanical support, it also delivers the radiation dose to block proliferation of the smooth muscle cells. Both of these methods are well-known in the art (see for example, Regal et al., Eur. Heart J., 23:1038-1044, 2002; Leon et al., N. Engl. J. Med., 2002; Weinberger, Herz 23:366-372, 1998, Suntharalingam et al., Int. J. Radiat. Oncol. Biol. Phys. 52:1075-1082, 2002).

Methods for Determining Efficacy

Human patients treated with the compounds of the present invention are typically followed by a physician to track the success of the treatment. Indications of treatment failure include patient complaints of symptoms returning, failure of standard stress tests, and return of disease indicators such as reduced blood flow, ischemia, and myocardial infarction. In addition, coronary angiography is often used to determine lumen diameter of the treated vessel. An increase of 25% or more, preferably 50% or more, and most preferably 75% or more in lumen diameter post treatment as compared to pre-treatment is indicative of therapeutic efficacy. The diameter of the treated vessel can also be compared to a reference distal and proximal segment to determine therapeutic efficacy. Additional methods for measuring therapeutic efficacy include magnetic resonance angiography and intravascular ultrasound (IVUS), which allows for quantitation of neointimal formation, luminal diameter, plaque area and volume. The patient is monitored both in the short-term (up to six months after initial treatment) and the long-term (six months or more after the initial treatment) to determine the efficacy of the treatment using the compounds of the present invention.

Dosages and toxicity for compounds can be measured in vivo using the animal models described below. For example, rat models of vascular injury by balloon catheterization can be used to test each compound as well as combinations of compounds for efficacy and toxicity (see Powell et al., J. Cardiovasc. Pharmacol., 16:S42-9, 1990). Tests for efficacy include measurement of neointimal formation as a percentage of cross-sectional measurements of neointima/media and arterial blood pressure measurements. In addition, an in vitro system using cultures of smooth muscle cells can be used to determine effective dosages of each compound as well as compound stability. For example, a specific number of cells can be plated in culture, stimulated with known mitogens such as growth factors or angiotensin II, and treated with increasing concentrations of each drug compound. Each day, the number of cells in the culture can be counted and proliferation measured (see Powell et al., supra). A positive result is considered to be a decrease in proliferation rate of at least 10%, preferably 25% and more preferably 50% or more as compared to cells treated with mitogen alone.

### Animal Studies

The method of treatment provided in the present invention also includes administering a therapeutically effective amount of an N-phenyl-2-pyrimidine derivative capable of inhibiting PDGFR biological activity and a PI3K pathway inhibitor compound to a warm-blooded animal suffering from vascular stenosis or restenosis, in a dose sufficient to prevent or reduce vascular stenosis or restenosis. Such animals may also be treated with a combination of an N-phenyl-2-pyrimidine derivative, a PI3K pathway inhibitor compound, and, when desired, at least one of the following: (i) an angiogenesis inhibitor, (ii) an anti-proliferative compound (iii) an immunosuppressive compound, (iv) an anti-migratory compound, (v) an anti-platelet agent, and (vi) an anti-fibrotic compound.

Warm-blooded animals, as used herein, include but are not limited to mice, dogs, pigs, baboons, rats, rabbits, and monkeys. Also included are any animal models of coronary angioplasty or arterial stenosis which can be used to study dosages and efficacy of treatment (see for example Garas et al., supra)

### EXAMPLES

### Example 1

### In Vivo Testing of Coated Stents in a Porcine Coronary Artery Model

This preliminary study is conducted to assess the ability of rapamycin released from ε-caprolactone-co-glycolide copolymer-coated stents to inhibit intimal hyperplasia in vivo. Fourteen days after receiving rapamycin-loaded or control polymer coated stents, the male-Yorkshire pigs are euthanized and the coronary arteries removed, the vessels prepared for histological evaluation and analyzed for the amount of intimal growth. Through comparison to control metal stents and stents containing polymer only, the in vivo ability of rapamycin to prevent neointimal growth can be determined.

Ethylene oxide-sterilized Palmaz-Schatz stents are implanted under sterile conditions in anesthetized farm pigs weighing 38 to 48 kg. Twenty-four hours prior to stent implantation, animals are given aspirin (325 mg, p.o., qd) and ticlopidine (250 mg, p.o., qd) to control chronic thrombosis; both aspirin and ticlopidine are continued daily until sacrifice. Anesthesia is induced with ketamine (20 mg/kg, i.m.), xylazine (2 mg/kg, i.m.) and sodium pentobarbital (10 mg/kg as needed) and maintained on 1-2% isofluorane in oxygen. An 8 Fr sheath is placed in an aseptically isolated left carotid artery and used subsequently to conduct either an 8 Fr JL 3.5 guide catheter for coronary angiography or to place a 0.014 inch guidewire for balloon delivery of stents to the appropriate coronary arteries. Heparin (150 unit/kg) is administered intraprocedurally to prevent acute thrombosis. Four experimental groups are employed; 1) metal stent control; 2) metal stent coated with 45/55 (w/w) ε-caprolactone glycolide copolymer (CAP/GLY); 3) 32 μg rapamycin/stent formulated in CAP/GLY; 4) 166 μg rapamycin/stent formulated in CAP/GLY. Stents are deployed in both the LAD and LCX coronary arteries. Angiography is performed prior to, during, and immediately after stenting to both size the vessel for choice of balloon diameter (3.0, 3.5 or 4.0 mm) and to obtain measurements for determination of the balloon/artery ratio. Stents are deployed by inflating the delivery balloon to 8-10 ATM for 30 seconds. Angiography is also performed at 14 days post-implantation to obtain final vessel diameter. Treatment groups are randomized and individual stents are implanted by an investigator who is blinded as to the treatment. However, only one treatment is employed in any given pig. Fourteen days after implantation, animals are euthanized, the vessels are perfusion fixed for ten minutes at 100 mmHg with 10% formalin and then stored in 10% buffered formalin.

For histological assessment, the stented vessel is embedded in glycol methacrylate. Four 3-5 μm thick cross-sections taken at equal intervals along the length of the stent are placed on glass slides and prepared with Miller's Elastin stain. Histomorphometric measurements are determined in each section via microscopy and computerized image analysis. The intima:media ratio is determined for each experimental group. A positive result is a reduction of the intima:media ratio of at least 10%, preferably 25%, and most preferably 50% or more as compared to control animals not receiving rapamycin treatment.

### Example 2

### Effects of Gleevec and Rapamycin on Smooth Muscle Cell Proliferation and Migration

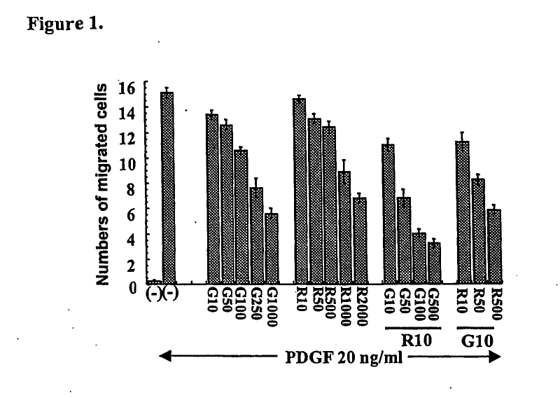

In vitro studies were performed to examine the dose response of Gleevec and rapamycin on human aortic vascular smooth muscle cells (HASOM cells). Cell migration was measured using a 24-well Boyden-chamber (Corning Costar Corp, Cambridge, Mass.). Each well had an insert containing a polycarbonate filter with eight μm pores. These membranes were coated with 0.1% gelatin for eight hours, then aspirated and allowed to dry for two hours. The cells were allowed to grow to 70% confluence and then treated with media containing 1% fetal calf serum and drug compounds (Gleevec alone, rapamycin alone and Gleevec and rapamycin together at the concentrations indicated in FIG. 1) for 48 hours prior to the migration assay. It should be noted that rapamycin does not block migration in this assay if the 48 hour incubation is not used (Poon et al., J. Clin. Invest. 98:2277-2283, 1996). The cells were harvested in 0.05% trypsin, resuspended in M-199 media, and seeded on the coated membrane at a concentration of 4000 cells per well with Gleevec and/or rapamycin. The M-199 media containing 0.5% BSA and 20 ng/ml human PDGF-B (hPDGF-BB) was placed in the bottom Boyden chamber. Cells were incubated in 5% CO₂ at 37° C. for 12 hours and the cells lying on the upper surface of the membrane were scraped. The membranes were removed and the cells on the lower surface of the membrane were stained with hematoxylin. Cell migration was quantified by counting the cells in five random high-power fields on the lower surface of the membrane. All assays were run in triplicate. The results of this assay demonstrated that Gleevec alone, rapamycin alone and the combination of Gleevec and rapamycin potently inhibited PDGF induced migration of HASOM cells in a dose dependent manner (FIG. 1). Moreover, the combination of Gleevec and rapamycin provided an enhanced inhibition of migration (FIG. 1). Note that the effective concentrations of rapamycin are well below those that typically cause immunosuppression (roughly 5-15 ng/ml).

Migration of smooth muscle cells is an important event in the pathogenesis of the neointimal hyperplastic lesion. These experiments demonstrate that Gleevec and rapamycin inhibit PDGF-BB/serum induced smooth muscle cell migration. It should also be noted that the maximum inhibition that we have seen to date is approximately 70-80%, which may be due to the fact that there are small amounts of serum in the assay that likely contains factors which induce signals not blocked by Gleevec or rapamycin. Extrapolating to the microenvironment of the angioplasty lesion, it is possible that the use of additional compounds, as described herein, could increase the inhibition of smooth muscle cell migration to a near maximal level.

### OTHER EMBODIMENTS

From the foregoing description, it is apparent that variations and modifications may be made to the invention described herein to adopt it to various usages and conditions. Such embodiments are also within the scope of the following claims.

All publications mentioned in this specification are herein incorporated by reference to the same extent as if each independent publication or patent application was specifically and individually indicated to be incorporated by reference.

What is claimed is:

1. A method for preventing or treating the occurrence of vascular stenosis or restenosis following angioplasty comprising administering to a patient (i) a first compound capable of inhibiting platelet derived growth factor receptor (PDGFR) biological activity and (ii) a PI3K pathway inhibitor compound, wherein said administering is in an amount and for a time sufficient to prevent or reduce the occurrence of stenosis or restenosis following angioplasty.

2. The method of claim 1, wherein said first compound is an N-phenyl-2-pyrimidine derivative.

3. The method of claim 2, wherein said N-phenyl-2-pyrimidine derivative is imatinib mesylate.

4. The method of claim 1, wherein said first compound inhibits PDGFR β biological activity.

5. The method of claim 1, wherein said first compound inhibits PDGFR biological activity stimulated by a PDGF-BB ligand.

6. The method of claim 1, wherein said PI3K pathway inhibitor compound inhibits the biological activity of any protein on the PI3K/Akt/mTOR signaling pathway.

7. The method of claim 6, wherein said compound inhibits the biological activity of mTOR.

8. The method of claim 7, wherein said compound is rapamycin.

9. The method of claim 1, wherein said restenosis is characterized by the migration of smooth muscle cells into the intima.

10. The method of claim 1, wherein said restenosis is characterized by the proliferation of vascular smooth muscle cells.

11. The method of claim 1, wherein said restenosis is characterized by the deposition of extracellular matrix.

12. The method of claim 1, wherein said restenosis follows angioplasty and the use of a stent for treatment.

13. The method of claim 12, wherein said stent is coated with a compound capable of inhibiting PDGFR biological activity and a PI3K pathway inhibitor compound.

14. The method of claim 13, wherein said compound capable of inhibiting PDGFR biological activity is imatinib mesylate and said PI3K pathway inhibitor compound is rapamycin.

15. The method of claim 1, wherein said vascular stenosis is treated with angioplasty and the use of a stent.

16. The method of claim 15, wherein said stent is coated with a compound capable of inhibiting PDGFR biological activity and a PI3K pathway inhibitor compound.

17. The method of claim 16, wherein said compound capable of inhibiting PDGFR biological activity is imatinib mesylate and said PI3K pathway inhibitor compound is rapamycin.

18. The method of claim 1, wherein said compound capable of inhibiting PDGFR biological activity and said PI3K pathway inhibitor compound are given in combination with a pharmaceutically acceptable carrier.

19. The method of claim 1, wherein said amount is sufficient to prevent or reduce vascular smooth muscle cell hyperplasia.

20. The method of claim 1, further comprising administering to a patient at least one additional compound selected from the group consisting of:
1) an angiogenesis inhibitor,
2) an anti-proliferative compound,
3) an immunosuppressive compound,
4) an anti-migratory compound,
5) an anti-platelet agent, and
6) an anti-fibrotic compound.

21. The method of claim 20, wherein said additional compound is an angiogenesis inhibitor selected from the group consisting of an antibody; an antibody that binds VEGF-A; an antibody that binds a VEGF receptor and blocks VEGF binding; avastin; endostatin; angiostatin; restin; tumstatin; TNP-470; 2-methoxyestradiol; thalidomide; a peptide fragment of an anti-angiogenic protein; canstatin; arrestin; a VEGF kinase inhibitor; CPTK787; SFH-1; an anti-angiogenic protein; thrombospondin-1; platelet factor-4; interferon-α; an agent that blocks TIE-1, TIE-2 or PIH12 signalling; an agent that blocks an extracellular vascular endothelial (VE) cadherin domain; an antibody that binds to an extracellular VE-cadherin domain; tetracycline; penicillamine; vinblastine; cytoxan; edelfosine; tegafur; uracil; curcumin; green tea; genistein; resveratrol; N-acetyl cysteine; captopril; a cox-2 inhibitor; celecoxib; and rofecoxib.

22. The method of claim 20, wherein said additional compound is an anti-proliferative compound selected from the group consisting of rapamycin; taxol; troglitazone; an agent that inhibits VEGF; an agent that inhibits bFGF; an antibody that binds bFGF-saporin; a statin; an ACE inhibitor; suramin; 17 beta-estradiol; atorvastatin; fluvastatin; lovastatin; pravastatin; simvastatin; cerivastatin; perindopril; quinapril; captopril; captopril; lisinopril; enalapril; fosinopril; cilazapril; ramipril; and a kinase inhibitor.

23. The method of claim 22, wherein said agent that inhibits VEGF is an antibody.

24. The method of claim 22, wherein said agent that inhibits bFGF is an antibody.

25. The method of claim 20, wherein said additional compound is an immunosuppressive compound selected from the group consisting of prednisone; FTY720; methylprednisolone; α-tocopherol; azathioprine; chlorambucil; cyclophosphamide; an antibody that binds to an IL-2 receptor or to CTLA4; methotrexate; mycophenolate mofetil; cyclosporine; an agent that interferes with macrophage function; an agent that inhibits P-selectin PSGL-1; VLA-4; VCAM-1 or Mac-1 biological function; and FTY720.

26. The method of claim 20, wherein said additional compound is an anti-migratory compound selected from the group consisting of cyproheptadine; endothelin receptor antagonist; serotonin receptor antagonist; methysergide; bosentan; YM087; cyproheptadine; ketanserin; and anplag.

27. The method of claim 20, wherein said additional compound is an anti-platelet agent selected from the group consisting of aspirin; ticlopidine; cilostazol; dipyridamole; abciximab; clopidogrel; dipyridimole; a glycoprotein iib/iiia inhibitor; an adenosine reuptake inhibitor; an ADP inhibitor; eptifibatide; tirofiban; a phosphodiesterase III inhibitor; and ticlopdipine.

28. The method of claim 20, wherein said additional compound is an anti-fibrotic compound selected from the group consisting of an agent that blocks TGF-β signaling or inhibits activation of plasminogen activator inhibitor-1 promoter activity; an antibody that binds to TGF-β or to a TGF-β receptor; an antibody that binds to TGF-β receptor I, II, or III; a kinase inhibitor; an agent that blocks CTGF signaling; an agent that inhibits prolyl hydroxylase; an agent that inhibits procollagen C-proteinase; pirfenidone; silymarin; pentoxifylline; colchicines; embrel; remicade; an agent that antagonizes TGF-β; an agent that antagonizes CTGF; and an agent that inhibits VEGF.

29-68. (canceled)
